(19) United States
(12) Patent Application Publication
(10) Pub. No.: US 2007/0104753 A1
(43) Pub. Date: May 10, 2007

(54) Medical device with a coating comprising an active form and an inactive form of therapeutic agent(s)

(76) Inventor: Aiden Flanagan, Galway (IE)

Correspondence Address: JONES DAY, 222 EAST 41ST ST, NEW YORK, NY, 10017, US

(21) Appl. No.: 11/267,598

(22) Filed: Nov. 4, 2005

(51) Int. Cl. A61K 31/4745; A61K 31/337; A61F 2/02

(52) U.S. Cl. 424/423; 514/291; 514/449

(57) ABSTRACT

The present invention relates generally to coated medical devices, such as a stent, that has a surface completely or partially covered with a coating that comprises a first region containing an inactive form of a therapeutic agent and a second region containing an active form of a therapeutic agent. The present invention also relates to medical device comprising a first coating composition containing an inactive therapeutic agent and a second coating composition comprising an active therapeutic agent.

### 1. FIELD OF THE INVENTION

The present invention relates generally to medical devices that are useful for delivering a therapeutic agent to a body tissue, such as a body lumen, and methods for making and using such medical devices. In particular, the invention is directed to a medical device having a surface and a coating disposed thereon. The coating comprises a first region including a first therapeutic agent in an inactive form and a second region including a second therapeutic agent in an active form. The coating is capable of providing sustained release of the active form of a therapeutic agent over a time period. The invention also relates to a coating comprising a first quantity of a coating composition containing an inactive form of a first therapeutic gent and a second quantity of a coating composition containing an active form of a second therapeutic agent.

### 2. BACKGROUND OF THE INVENTION

Cardiovascular disease is a leading cause of death in the developed world. Patients having such disease usually have narrowing or closing (stenosis) in one or more arteries. Medical devices, such as implantable stents, have been used for delivering therapeutic agents, e.g. drugs, to body tissue such as a body lumen. Various types of drug-coated stents have been used for the localized delivery of drugs to the wall of a body lumen to prevent restenosis. However, it has been shown in the tailored drug TDP that when a device comprising a coating containing a therapeutic agent is deployed in the body, only a relatively small amount of therapeutic agent or drug is released from the region at or near the surface of the coating, leaving a majority of the drug in the coating. It was found that close to 100% of the drug that is released from the coating was located in a limited region at or near the outer surface of the coating. The thickness of this region is generally about 10-20% of the entire coating thickness.

In the medical device industry, there are some concerns about leaving a significant amount of unreleased therapeutic agent in the coating for the lifetime of an implanted medical device. In order to confine the therapeutic agent to the outermost region of the coating and to reduce the amount of unreleased therapeutic agent remaining in the coating, attempts have been made to provide two layers of coating material on the medical device surface. In particular, attempts have been made to achieve this by coating a first polymer layer which does not include a therapeutic agent and then applying a second coating layer on top of the first polymer layer, in which the second coating layer includes a therapeutic agent. These medical devices may be coated by various methods with compositions that comprise one or more therapeutic agents. For example, spraying is a common technique for applying a coating uniformly to a surface of a medical device. However, this process requires two coating steps which reduces its economical efficiency.

Accordingly, there is a need for more efficient and cost-effective methods of delivering a therapeutic agent to a targeted body tissue as well as reducing the residual amount of unreleased therapeutic agent in the coating. There is also a need to provide a coated medical device and a simpler method of manufacturing the coated medical device for delivering a therapeutic agent which coating does not retain a significant residual or unreleased amount of the therapeutic agent.

### 3. SUMMARY OF THE INVENTION

The present invention is directed to a medical device comprising a surface and a coating disposed on at least a portion of the surface wherein the coating comprises a first region comprising a first amount of an inactive form of a first therapeutic agent and a second region situated over at least a portion of the first region, wherein the second region comprises a second amount of an active form of a second therapeutic agent. In specific embodiments, the second region is situated adjacent to the first region. In other specific embodiments, the first and second therapeutic agents are the same. In other specific embodiments, the first and second therapeutic agents are different.

In a specific embodiment, the coating further comprises a third region. In certain embodiments, the third region is situated between the first region and the second region, and wherein the third region comprises the active form of the second therapeutic agent and the inactive form of the first therapeutic agent. In specific embodiments, the third region is situated over at least a portion of the second region. In other embodiments, the third region is situated under the first region. In certain embodiments, the first region is a first layer and the second region is a second layer situated over at least a portion of the first layer. In certain embodiments, the coating further comprises a third layer. In specific embodiments, the third layer is situated between the first and second layers. In other embodiments, the third layer is situated over at least a portion of the second layer. In other specific embodiments, the third layer is situated under the first layer.

The present invention is also directed to a stent comprising a surface and a coating disposed on at least a portion of the surface, wherein the coating comprises a first region comprising a first amount of an inactive form of a therapeutic agent and a second region situated over at least a portion of the first region, wherein the second region comprises a second amount of an active form of the therapeutic agent and wherein the therapeutic agent inhibits smooth muscle cell proliferation, contraction, migration or hyperactivity.

The present invention is further directed to a medical device comprising a surface and a coating disposed on at least a portion of the surface, wherein the coating comprises a first quantity of a first coating composition in which the first quantity comprises a first amount of an inactive form of a first therapeutic agent, and a second quantity of a second coating composition disposed over at least a portion of the first quantity, wherein the second quantity comprises a second amount of an active form of a second therapeutic agent.

The present invention is also directed to an implantable stent comprising a metallic intravascular balloon-expandable open lattice sidewall stent structure designed for permanent implantation into a blood vessel of a patient; and a coating conforming to the open lattice sidewall so as to preserve the open lattice sidewall structure of the stent, wherein the coating comprises a first quantity of a first coating composition comprising a first amount of an inactive form of a therapeutic agent; and a second quantity of a second coating composition disposed over at least a portion of the first quantity, wherein the second coating composition comprises a second amount of an active form of the therapeutic agent, and wherein the therapeutic agent inhibits smooth muscle cell proliferation, contraction, migration or hyperactivity.

The present invention is further directed to a method of making a medical device comprising a surface, the method comprises: (a) disposing a coating composition comprising an active form of a therapeutic agent on at least a portion of the surface to form a coating thereon; and (b) exposing the coating to energy generated by an energy source to inactivate the therapeutic agent in a first region of the coating while allowing the therapeutic agent in a second region of the coating to remain in the active form. In a specific embodiment, the second region is situated over at least a portion of the first region. In another embodiment, the first region is disposed adjacent to at least a portion of the surface. In a specific embodiment, the energy is more readily absorbed by the medical device than the coating. In another embodiment, the absorption of the energy by the medical device causes an increase in temperature at the surface.

The present invention is also directed to a method of making a medical device comprising a surface, the method comprises: (a) disposing a coating composition comprising an inactive form of a therapeutic agent on at least a portion of the surface to form a coating thereon; and (b) exposing the coating to an activation energy to activate the therapeutic agent in a first region of the coating while allowing the therapeutic agent in a second region of the coating to remain in the inactive form.

A method of making a medical device comprising a surface, said method comprises: (a) disposing a first quantity of a first coating composition on at least a portion of the surface, wherein the first quantity comprises a first amount of an inactive form of a first therapeutic agent; and (b) disposing a second quantity of a second coating composition on the first quantity, wherein the second quantity comprises a second amount of an active form of a second therapeutic agent.

As used herein, the term “therapeutic agent” includes biologically active materials, such as pharmaceuticals, drugs, genetic materials, and biological materials.

As used herein, the term “active form of a therapeutic agent” refers to a therapeutic agent that exhibits a desired biological or pharmaceutical effect. In certain embodiments the active form of the therapeutic agent may lose its desired activity or become inactivated.

As used herein, the term “inactive form of a therapeutic agent” refers to a therapeutic agent that is damaged or modified chemically/biologically that renders it inactive or it no longer exhibits a desired biological or pharmaceutical effect. In certain embodiments, the inactive form of the therapeutic agent may gain a desired activity or become activated. In specific embodiments, the inactive form of the therapeutic agent is a prodrug.

As used herein, the term “prodrug” refers to a drug which is in an inactive (or significantly less active) form. The prodrug can be metabolized in the body (in vivo) into the active form. In specific embodiments, the prodrug is a derivative of a biologically active material that can hydrolyze, oxidize, or otherwise react under biological conditions (in vitro or in vivo). Although a prodrug may become active when such reactions occur, the prodrug may have certain activity in its unreacted form. Examples of prodrugs that are useful in this invention include, but are not limited to, analogs or derivatives of a biologically active material that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include derivatives of a biologically active material that comprise —NO, —NO₂, —ONO, or —ONO₂ moieties. Prodrugs can typically be prepared using well-known methods, such as those described by BURGER'S MEDICINAL CHEMISTRY AND DRUG DISCOVERY (1995) 172-178, 949-982 (Manfred E. Wolff ed., 5th ed) and Design of Prodrugs (H. Bundgaard ed., Elselvier, N.Y. 1985).

As used herein, the term “therapeutically effective amount” refers to that amount of the therapeutic agent sufficient to delay or minimize the onset of symptoms such as for example those associated with cell proliferation, contraction, migration, hyperactivity, or address other conditions. A therapeutically effective amount may also refer to the amount of the therapeutic agent that provides a therapeutic benefit in the treatment or management of certain conditions such as for example stenosis or restenosis and/or the symptoms associated With stenosis or restenosis.

As used herein, the terms “subject” and “patient” are used interchangeably. As used herein, a subject is preferably a mammal such as a non-primate (e.g., cows, pigs, horses, cats, dogs, rats, etc.) and a primate (e.g., monkey and human), most preferably a human.

### 4. BRIEF DESCRIPTION OF FIGURES

FIG. 1 shows an embodiment of a medical device having a coating on the surface. The coating comprises a first region containing an inactive form of a first therapeutic agent and a second region containing an active form of a second therapeutic agent. The final and second therapeutic agents may be the same or different.

FIG. 2 shows a medical device having a coating on its surface. The coating comprises a first region containing an inactive form of a first therapeutic agent and a second region containing an active form of a second therapeutic agent. The first and second therapeutic agent may be the same or different. In this embodiment, an intermediate region is also present which contains both the inactive form of the first therapeutic agent and the active form of the second therapeutic agent.

FIG. 3A shows a medical device having a coating on the surface. The coating comprises: (i) a first region containing an inactive form of a first therapeutic agent; (ii) a second region containing an active form of a second therapeutic agent; and (iii) a third region situated over the second region.

FIG. 3B shows a medical device having a coating which comprises: (i) a first region containing an inactive form of a first therapeutic agent; and (ii) a second region containing an active form of a second therapeutic agent; and (iii) a third region situated under the first region.

FIG. 4A shows a medical device having a coating comprising: (i) a first coating layer comprising an inactive form of a first therapeutic agent; and (ii) a second coating layer comprising an active form of a second therapeutic agent.

FIG. 4B shows a medical device having a coating comprising: (i) a first coating layer comprising an inactive form of a first therapeutic agent; (ii) a second coating layer comprising an active form of a second therapeutic agent; and (iii) a third coating layer situated under the first coating layer.

FIG. 4C shows a medical device having a coating comprising: (i) a first coating layer comprising an inactive form of a first therapeutic agent; (ii) a second coating layer comprising an active form of a second therapeutic agent; and (iii) a third coating layer situated over the second coating layer.

FIGS. 5A-B shows an embodiment of a method of making a medical device of the present invention.

FIG. 6 is a cross-section of a stent strut with a coating showing a temperature gradient within the coated stent.

FIGS. 7A-C shows another embodiment of a method of making a medical device of the present invention.

FIGS. 8A-D shows another embodiment of a method of making a medical device of the present invention.

### 5. DETAILED DESCRIPTION OF ILLUSTRATIVE EMBODIMENTS

For clarity of disclosure, and not by way of limitation, the detailed description is divided into the subsections which follow.

### 5.1 Coated Medical Devices

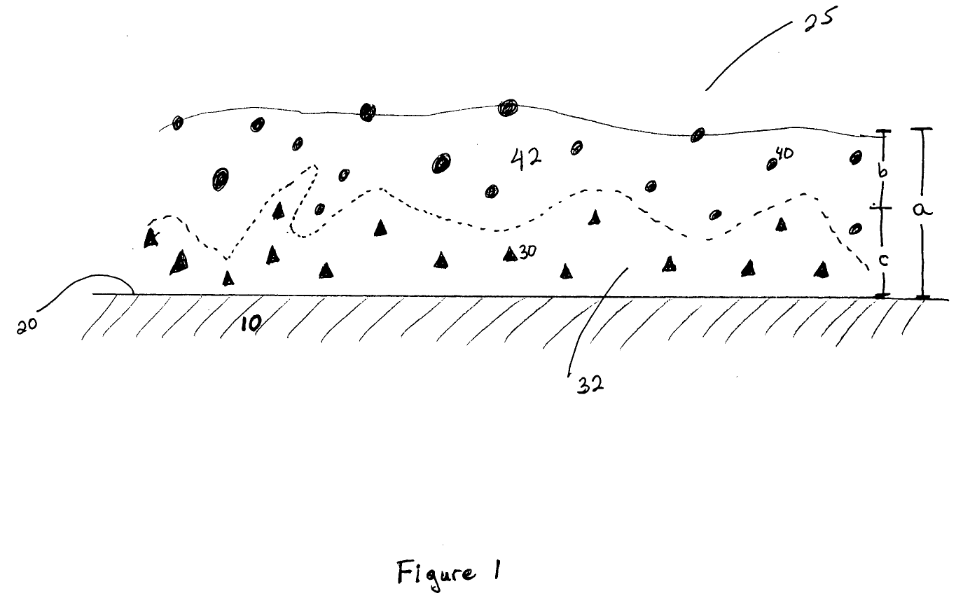

In one embodiment of the present invention, a coating is disposed on a surface of a medical device. The coating comprises a first region containing an inactive form of a first therapeutic agent and a second region containing an active form of a second therapeutic agent. FIG. 1 depicts a cross-sectional view of a medical device 10 having a surface 20 that can be implantable inside a subject. The surface 20 is covered at least in part, with a coating 25 which comprises a first region 32 that is disposed on the surface 20 of the medical device 10, and a second region 42 that is situated over the first region 32. Although the second region is situated over the first region, the second region need not be deposited or formed on the first region. As discussed below, the first and second regions can be formed when a single coating composition is applied to the surface of a medical device. In certain embodiments the second region is situated adjacent to the first region as shown in FIG. 1. The first region 32 comprises a first amount of an inactive form of a first therapeutic agent 30. The second region 42 comprises a second amount of an active form of a second therapeutic agent 40. The first and second therapeutic agent can be the same or different.

The first and second regions may be defined in terms of weight percent of the coating. Such weight percents can be measured preferably when coating is dried or the solvent used to make the coating composition has evaporated. The first region 32 can be at least 1-10%, at least 10-20%, at least 20-30%, at least 30-40%, at least 40-50%, at least 50-60%, at least 60-70%, at least 70-80%, at least 80-90%, or at least 90-99% by weight of the coating 25. Preferably, the first region comprises about 50% to about 95% weight percent of the coating. More preferably the first region comprises about 80% to about 90% weight percent of the coating. In certain embodiments, the second region 42 is at least 1-10%, at least 10-20%, at least 20-30%, at least 30-40%, at least 40-50%, at least 50-60%, at least 60-70%, at least 70-80%, at least 80-90%, or at least 90-99% by weight of the coating 25. Preferably, the second region 42 comprises about 5% to about 50% weight percent of the coating 25. More preferably the second region 42 comprises about 10% to about 20% weight percent of the coating 25.

The first region 32 has an average thickness of c. The second region 42 has an average thickness of b. The average thickness of the coating is a. The average thickness of a coating or a region is determined by taking measurements of the thickness of the coating or region taken at various points and taking the average of those measurements. The dashed line in FIG. 1 indicates an interface where the first region 32 and the second region 42 meet. The interface may or may not be a distinct interface.

The average thickness of the first region 32 and the average thickness of the second region 42 may be defined in terms of percent of average thickness of the coating 25. The average thickness, c, of the first region 32 is at least 1-10%, at least 10-20%, at least 20-30%, at least 30-40%, at least 40-50%, at least 50-60%, at least 60-70%, at least 70-80%, at least 80-90%, or at least 90-99% of the average thickness, a, of the coating 25. Preferably, the average thickness of the first region is about 50% to about 95% of the average thickness of the coating. In other embodiments, the average thickness, b, of the second region 42 is at least 1%, at least 1-10%, at least 10-20%, at least 20-30%, at least 30-40%, at least 40-50%, at least 50-60%, at least 60-70%, at least 70-80%, at least 80-90%, or at least 90-99% of the average thickness, a, of the coating 25. Preferably the average thickness of the second region is about 5% to about 50% of the average thickness of the coating.

Also, the first region may be at least 0.001-0.1 micron, at least 0.1-0.5 micron, at least 0.5-1 micron, 1-2 microns, 2-4 microns, 4-6 microns, 6-8 microns, 8-10 microns or 10-20 microns in thickness. In other embodiments, the second region may be at least 0.1-0.5 microns, at least 0.5-1 micron, 1-2 microns, 2-4 microns, 4-6 microns, 6-8 microns, 8-10 microns in thickness. The average thickness of the first or second region can each be from about 0.001 to about 100 microns; preferably the thickness can be from about 1 to about 10 microns.

The first and second regions are formed from a coating composition comprising a certain amount or dosage of therapeutic agents. Coating compositions suitable for forming the coating of the medical devices of the present invention can include one or more therapeutic agents as described in Section 5.3 infra as well as one or more polymers as described in Section 5.4 infra. In specific embodiments, the coating comprises at least 1-5%, at least 5-10%, at least 10-20%, at least 20-30%, at least 30-40%, at least 40-50%, at least 50-60%, at least 60-70, at least 70-80%, at least 80-90%, or at least 90-99% by weight of an active form of the second therapeutic agent. Preferably, the coating comprises about 0.5% to about 18% by weight of the active form of the second therapeutic agent. More preferably the coating comprises about 0.8% to about 7% by weight of an active form of the second therapeutic agent. In specific embodiments, the coating comprises at least 1-5%, at least 5-10%, at least 10-20%, at least 20-30%, at least 30-40%, at least 40-50%, at least 50-60%, at least 60-70, at least 70-80%, at least 80-90%, or at least 90-99% by weight of an inactive form of a therapeutic agent. Preferably, the coating comprises about 4% to about 35% by weight of the inactive form of the first therapeutic agent. More preferably the coating comprises about 4% to about 9% by weight of the inactive form of the first therapeutic agent.

In certain embodiments, the first region comprises the inactive form of the first therapeutic agent in an amount of about 0.5 μg/mm² to about 4 μg/mm² and preferably in an amount of about 0.8 μg/mm² to about 3.6 μg/mm² In some embodiments, the second region comprises the active form of the second therapeutic agent in an amount of about 0.05 μg/mm² to about 2 μg/mm² and preferably in an amount of about 0.1 μg/mm² to about 0.8 μg/mm². In specific embodiments, the ratio of the first amount of the inactive form of the first therapeutic agent to the second amount of the active form of the second therapeutic agent is at least 10:90, at least 20:80, at least 30:70, at least 40:60, at least 50:50, at least 60:40, at least 70:30, at least 80:20, or at least 90:10. Preferably this ratio is about 50:50 to about 95:5. More preferably the ratio is about 80:20 to about 90:10.

In certain embodiments, the first and second regions each comprises more than one therapeutic agents. The therapeutic agents in each region may be at the same amount or different amounts. In specific embodiments, the ratio of the amount of one therapeutic agent in a region to the amount of another therapeutic agent in the region is at least 10:90, at least 20:80, at least 30:70, at least 40:60, at least 50:50, at least 60:40, at least 70:30, at least 80:20, or at least 90:10. Preferably this ratio is about 10:90 to about 50:50. More preferably the ratio is about 20:80 to about 50:50.

In certain embodiments the first region comprises the active form of the second therapeutic agent in an amount of less than 1 weight percent of the region. In another embodiment, the second region comprises the inactive form of the first therapeutic agent in an amount of less than 1 weight percent of the region.

In certain embodiments, the coating comprises a polymer. Examples of suitable polymers are discussed below. In specific embodiments, the amount of polymer in the coating is at least 1-5%, at least 5-10%, at least 10-20%, at least 20-30%, at least 30-40%, at least 40-50%, at least 50-60%, at least 60-70, at least 70-80%, at least 80-90%, or at least 90-99% by weight of the coating. Preferably the coating comprises an amount of one or more polymer that is about 65 to about 92 weight percent of the coating.

In certain embodiments, the first and/or second regions can comprise one or more polymers. The polymers in each region may be at the same or different. For instance, the first region can comprise a first polymer and the second region can comprise a second polymer that is different from the first polymer. The second region can also comprise a third polymer that is the same as the first polymer.

In a specific embodiment, the coating is capable of releasing the active form of a therapeutic agent at a faster rate than the inactive form of the therapeutic agent. Preferably, the coating is capable of releasing the active form of a therapeutic agent at a rate that is at least about fifty times, at least about twenty times, at least about ten times, at least about five times, or at least about two times faster than the release of the inactive form of the therapeutic agent.

In another specific embodiment, the coating is capable of releasing a higher amount of the active form of a therapeutic agent than the inactive form of the therapeutic agent. Preferably, the coating is capable of releasing at least about fifty times, at least about twenty times, at least about ten times, at least about five times, or at least about two times the amount of the active form of a therapeutic agent than the inactive form of the therapeutic agent.

In certain embodiments, the coating is capable of providing sustained release of an active therapeutic agent over a time period. The time period for sustained release of the active form of a therapeutic agent from the coating can be at least about 30 minutes, at least about 30 minutes to 1 hour, at least about 1-2 hours, at least about 2-3 hours, at least about 3-4 hours, at least about 4-5 hours, at least about 5-6 hours, at least about 6-12 hours, at least about 12 hours-24 hours, at least about 1-2 days, at least about 2-3 days, at least about 3-4 days, at least about 4-5 days, at least about 5-6 days, at least about 6 days to 1 week, at least about 1-2 weeks, at least about 2-3 weeks, at least about 3 weeks to 1 month, at least about 1-2 months, at least about 2-3 months, at least about 3-4 months, at least about 4-5 months, at least about 5-6 months, at least about 6 months to 1 year, at least about 1-2 years, or longer. Preferably, the time period for sustained release of the active form of a therapeutic agent from the coating ranges from about 1 month to about 1 year, more preferably, from about 1 month to about 6 months.

In certain embodiments, the release rate of a therapeutic agent from a coating may be altered. For example, the release rate may be altered by using a laser to swell or modify the polymer matrix in a coating. Alternatively, release rate may be altered by shrinking the coating. For example, a polymer moiety that is cross-linkable when exposed to light, such as an UV curable glue, can be included as a component of the coating. Exposing this coating to light of different wavelengths causes the cross-linkable polymer in the coating to shrink, and to affect the release rate of the therapeutic agent in the coating. The release rate of a coating may also be altered by using a porous coating. For example, the release rate of a coating may be altered by incorporating unstable azine-type molecules to a coating. Such coating, when exposed to a gasification source, such as vibrations, sound waves or ultrasonic agitation, causes spontaneous gasification of the azine-type molecules. The gasification of the azine-type molecules creates pores in the coating which alters the release rate of the therapeutic agent in the coating.

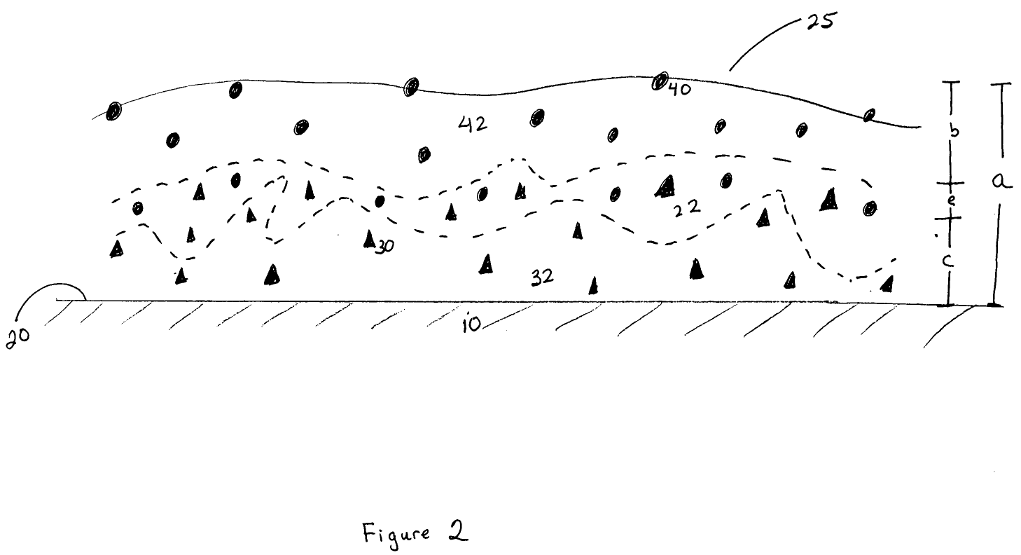

FIG. 2 depicts another embodiment of the present invention. A medical device 10 having a surface 20 is covered with a coating 25 which comprises a first region 32 that is adjacent to the surface 20 of the medical device, and a second region 42 that is situated over the first region. The coating 25 further comprises a third region, in this case an intermediate region 22, which comprises an active form of a therapeutic agent 40 and an inactive form of a therapeutic agent 30. The intermediate region 22 is situated between the first and the second region. The dashed lines show where the regions meet. The intermediate region 22 may be formed inadvertently or intentionally. The intermediate region has an average thickness e.

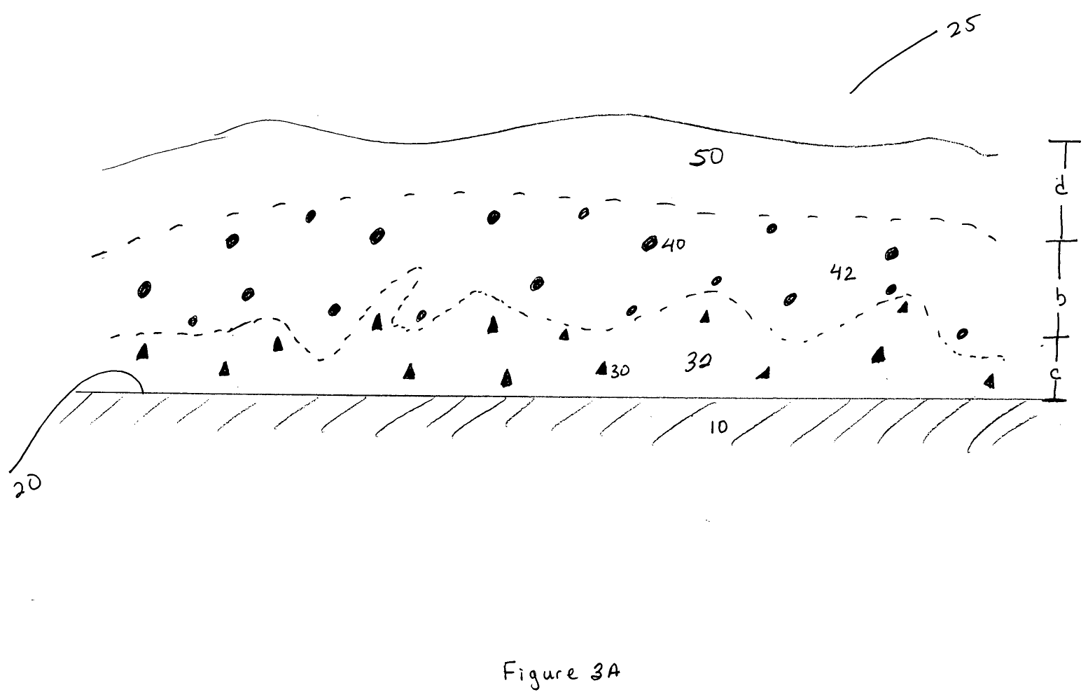

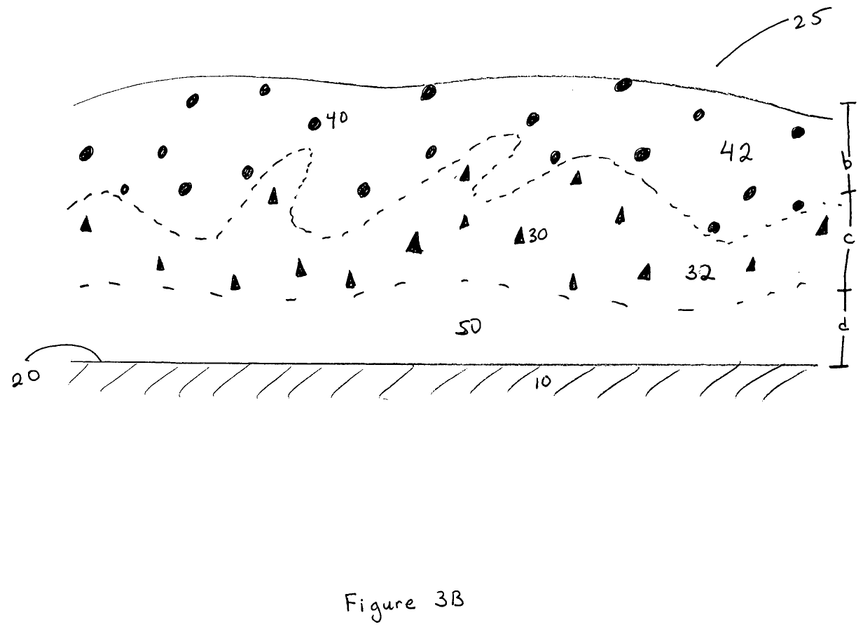

FIGS. 3A and 3B illustrate other embodiments of the present invention. In these embodiments coating further comprises a third region 50 having a thickness d, the third region 50 may or may not contain a therapeutic agent. In some embodiments, the third region comprises the active form of a therapeutic agent in an amount of less than 1 weight percent of the third region. The third region can be situated over the second region as indicated in FIG. 3A or it can be situated under the first region 32 indicated in FIG. 3B. In other embodiments, more than three regions may be present and each region may comprise a therapeutic agent and/or a polymer or other materials described in Sections 5.4 and 5.5.

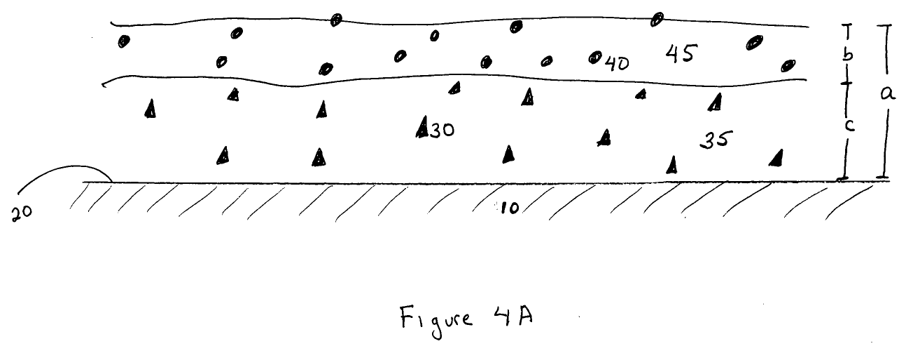

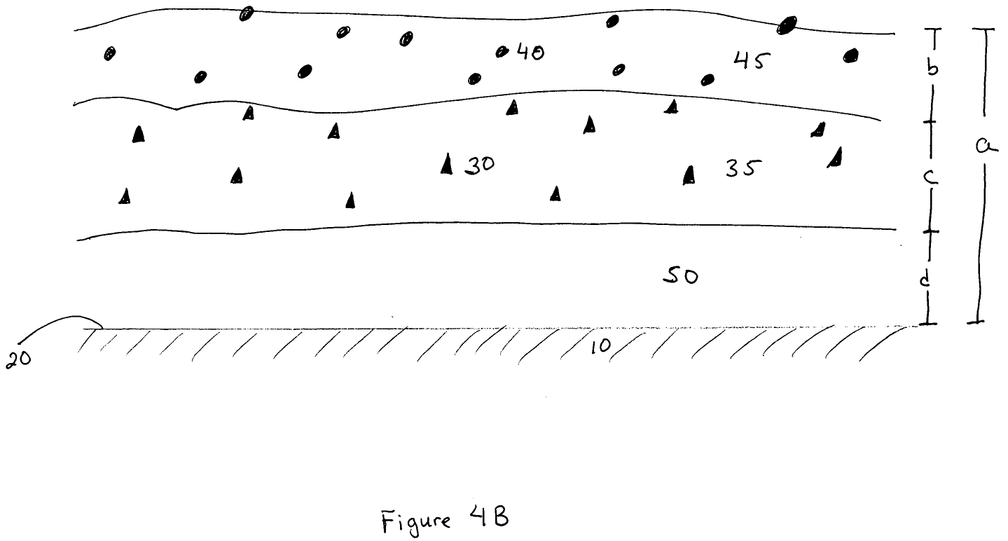

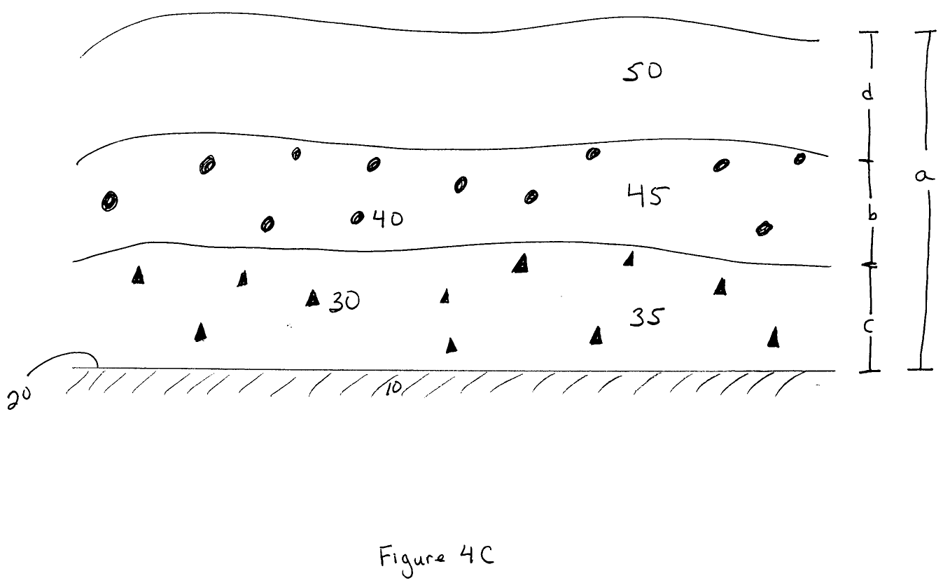

In certain embodiments, the regions of the coatings have relatively uniform thicknesses and can be considered layers. FIG. 4A illustrates one such embodiment of the present invention. A medical device 10 having a surface 20 is covered with a first coating layer 35, having a thickness c, which comprises an inactive form of a first therapeutic agent 30. A second coating layer 45, having a thickness b, which comprises an active form of a second therapeutic agent 40 is situated over at least a portion of the first coating layer 35. The first and second therapeutic agent can be the same or different.

FIGS. 4B and 4C illustrates embodiments where the coating comprises a third layer 50 with an average thickness of d. The third layer 50 can be situated under the first layer 35 as in FIG. 4B or over the second layer 45 as in FIG. 4C. Although only three layers are shown, the coating can include additional layers.

In another embodiment, the medical device of the present invention comprises a surface and a coating disposed on at least a portion of the surface. The coating comprises a first quantity of a first coating composition, in which the first quantity comprises a first amount of an inactive form of a first therapeutic agent. A second quantity of a second coating composition is disposed on at least a portion of the first quantity of first coating composition. The second quantity comprises a second amount of an active form of a second therapeutic agent. The first and second therapeutic agent can be the same or different. Preferably, the second quantity is disposed adjacent to the first quantity.

The first and second quantities may be defined in terms of weight percent of the coating. Such weight percents can be measured preferably when coating is dried or the solvent used to make the coating composition has evaporated. The first quantity can be at least 1-10%, at least 10-20%, at least 20-30%, at least 30-40%, at least 40-50%, at least 50-60%, at least 60-70%, at least 70-80%, at least 80-90%, or at least 90-99% by weight of the coating. Preferably, the first quantity comprises about 50% to about 95% weight percent of the coating. More preferably the first quantity comprises about 50% to about 60% weight percent of the coating. In certain embodiments, the second quantity is at least 1-10%, at least 10-20%, at least 20-30%, at least 30-40%, at least 40-50%, at least 50-60%, at least 60-70%, at least 70-80%, at least 80-90%, or at least 90-99% by weight of the coating. Preferably, the second quantity comprises about 5% to about 50% weight percent of the coating. More preferably the second quantity comprises about 40% to about 50% weight percent of the coating.

The first and second quantities comprise certain amounts or dosages of therapeutic agents. In specific embodiments, the coating comprises at least 1-5%, at least 5-10%, at least 10-20%, at least 20-30%, at least 30-40%, at least 40-50%, at least 50-60%, at least 60-70, at least 70-80%, at least 80-90%, or at least 90-99% by weight of an active form of the second therapeutic agent. Preferably, the coating comprises about 0.5 to about 18 percent by weight of the active form of the second therapeutic agent. More preferably the coating comprises about 0.8 to about 7 percent by weight of an active form of the second therapeutic agent. In specific embodiments, the coating comprises at least 1-5%, at least 5-10%, at least 10-20%, at least 20-30%, at least 30-40%, at least 40-50%, at least 50-60%, at least 60-70, at least 70-80%, at least 80-90%, or at least 90-99% by weight of an inactive form of a therapeutic agent. Preferably, the coating comprises about 4 to about 35 percent by weight of the inactive form of the first therapeutic agent. More preferably the coating comprises about 4 to about 9 percent by weight of the inactive form of the first therapeutic agent.

In certain embodiments, the first quantity comprises the inactive form of the first therapeutic agent in an amount of about 0.5 μg/mm² to about 3.8 μg/mm² and preferably in an amount of about 0.8 μg/mm² to about 3.6 μg/mm². In some embodiments, the second quantity comprises the active form of the second therapeutic agent in an amount of about 0.05 μg/mm² to about 2 μg/mm² and preferably in an amount of about 0.1 μg/mm² to about 0.8 μg/mm². In specific embodiments, the ratio of the first amount of the inactive form of the first therapeutic agent to the second amount of the active form of the second therapeutic agent is at least 10:90, at least 20:80, at least 30:70, at least 40:60, at least 50:50, at least 60:40, at least 70:30, at least 80:20, or at least 90:10. Preferably this ratio is about 50:50 to about 95:5. More preferably the ratio is about 80:20 to about 90:10.

In certain embodiments the first quantity comprises the active form of the second therapeutic agent in an amount of less than 1 weight percent of the first quantity. In another embodiment, the second quantity comprises the inactive form of the first therapeutic agent in an amount of less than 1 weight percent of the second quantity.

In certain embodiments, the coating comprises a polymer. Examples of suitable polymers are discussed below. In specific embodiments, the amount of polymer in the coating is at least 1-5%, at least 5-10%, at least 10-20%, at least 20-30%, at least 30-40%, at least 40-50%, at least 50-60%, at least 60-70, at least 70-80%, at least 80-90%, or at least 90-99% by weight of a polymer. Preferably the coating comprises an amount of one or more polymer that is about 65 to about 92 weight percent of the coating.

In certain embodiments, the first and/or second quantities can comprise one or more polymers. The polymers in each quantity may be at the same or different. For instance, the first quantity can comprise a first polymer and the second quantity can comprise a second polymer that is different from the first polymer. The second quantity can also comprise a third polymer that is the same as the first polymer.

In a specific embodiment, the coating is capable of releasing the active form of a therapeutic agent at a faster rate than the inactive form of the therapeutic agent. Preferably, the coating is capable of releasing the active form of a therapeutic agent at a rate that is at least about fifty times, at least about twenty times, at least about ten times, at least about five times, or at least about two times faster than the release of the inactive form of the therapeutic agent.

In another specific embodiment, the coating is capable of releasing a higher amount of the active form of a therapeutic agent than the inactive form of the therapeutic agent. Preferably, the coating is capable of releasing at least about fifty times, at least about twenty times, at least about ten times, at least about five times, or at least about two times the amount of the active form of a therapeutic agent than the inactive form of the therapeutic agent.

In certain embodiments, the coating is capable of providing sustained release of an active therapeutic agent over a time period. The time period for sustained release of the active form of a therapeutic agent from the coating can be at least about 30 minutes, at least about 30 minutes to 1 hour, at least about 1-2 hours, at least about 2-3 hours, at least about 3-4 hours, at least about 4-5 hours, at least about 5-6 hours, at least about 6-12 hours, at least about 12 hours-24 hours, at least about 1-2 days, at least about 2-3 days, at least about 3-4 days, at least about 4-5 days, at least about 5-6 days, at least about 6 days to 1 week, at least about 1-2 weeks, at least about 2-3 weeks, at least about 3 weeks to 1 month, at least about 1-2 months, at least about 2-3 months, at least about 3-4 months, at least about 4-5 months, at least about 5-6 months, at least about 6 months to 1 year, at least about 1-2 years, or longer. Preferably, the time period for sustained release of the active form of a therapeutic agent from the coating ranges from about 1 month to about 1 year, more preferably, from about 1 month to about 6 months.

### 5.2 Methods of Preparing and Using Coated Medical Devices

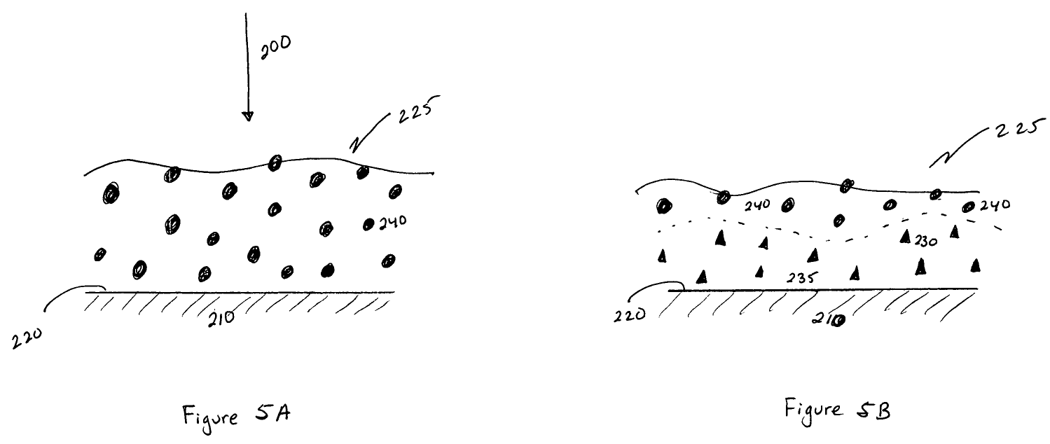

In one embodiment for preparing a medical device of the present invention, a coating composition comprising an active form of a therapeutic agent is disposed on a surface of a medical device to form a coating. The coating is then exposed to an energy source to inactivate the therapeutic agent in a first region of the coating, i.e. cause the active form of the therapeutic agent to lose its desired activity. The therapeutic agent in a second region of the coating remains in the active form. FIGS. 5A-5B illustrates an embodiment of this method. In FIG. 5A, a surface 220 of a medical device 210 is covered at least in part with a coating 225. The coating 225 comprises an active form of a therapeutic agent 240. Energy from an energy source 200, such as a laser beam, is applied to the coating 225 to inactivate the active form of the therapeutic agent 240. FIG. 5B shows the exposed coating having a first region 235 containing the inactive form of the therapeutic agent 230 and a second region of the coating 245 containing the active form of the therapeutic agent 240. The dashed line indicates where the two regions meet. Preferably, the second region 245 is situated over a portion of the first region 235 and/or the first region 235 containing the inactive therapeutic agent 230 is adjacent to at least a part of the medical device surface 220.

Before applying the coating composition to the surface of the medical device, the surface may optionally be subjected to a pre-treatment to enhance the adhesion of the coating to the surface. Such pre-treatment may include without limitation roughening of the surface, oxidizing the surface, or priming the surface.

To prepare the coating compositions suitable for the methods of the invention, a therapeutic agent, such as those described in Section 5.3 infra., is dissolved or suspended in a solvent. The coating compositions may also include one or more polymers such as those described in Section 5.4 infra. Suitable solvents for forming the coating composition are those that do not alter or adversely impact the properties of the therapeutic agent. Examples include without limitation tetrahydrofuran, chloroform, toluene, acetone, isooctane, or 1,1,1-trichloroethane. In the case that a polymer is included in the coating composition, it is preferable that the solvent be able to dissolve or suspend the polymer.

The coating composition may be applied to the surface of the medical device by methods that are known to the skilled artisan. Examples of suitable methods include without limitation spraying, dipping, brushing, swabbing, rolling, or electrostatic deposition. Preferably, the coating composition is applied to a surface of a medical device by spraying coating. More than one coating method can be used to apply the coating composition to the surface of the medical device.

In some embodiments it is preferable to apply the coating composition so that the openings in the medical device are preserved or not occluded. For instance, in the case of a stent having a sidewall with openings, it may be desirable to apply a conformal coating to the stent surface that does not occlude the openings, i.e., the coating conforms to the surface so that coating is not present in the openings.

Suitable energy sources are ones that emit energy or heat. Examples of such sources include without limitation lasers; high powered flash lamps, e.g. xenon lamps; electro-magnetic induction heaters, e.g. RF induction heaters; microwave, acoustic wave and ultrasonic wave. It is preferable that the energy emitted from the energy source have a wavelength of about 300 to about 5000 nanometers. Most preferably, the wavelength is about 1000 nanometers. In some embodiments, the wavelength is at least 700-800 nanometers, 800-900 nanometers, 900-1,000 nanometers, 1,000-1,200 nanometers, or 1,200-1,500 nanometers.

The energy source can be applied to the coating preferably for a duration of about 1 nanosecond to about 10 seconds, and more preferably for a duration of about 1 millisecond to about 1 second. In certain embodiments, the energy is applied for at least 10 seconds, at least 10-30 seconds, at least 30 seconds to at least 1 minutes, at least 1-2 minutes, and repeated at least 2 times, at least 2-5 times, or at least 5 to 50 times. Also, the energy can be applied to the coating more than once. Furthermore, the energy can be pulsated so that the coating is not continuously exposed to such energy.

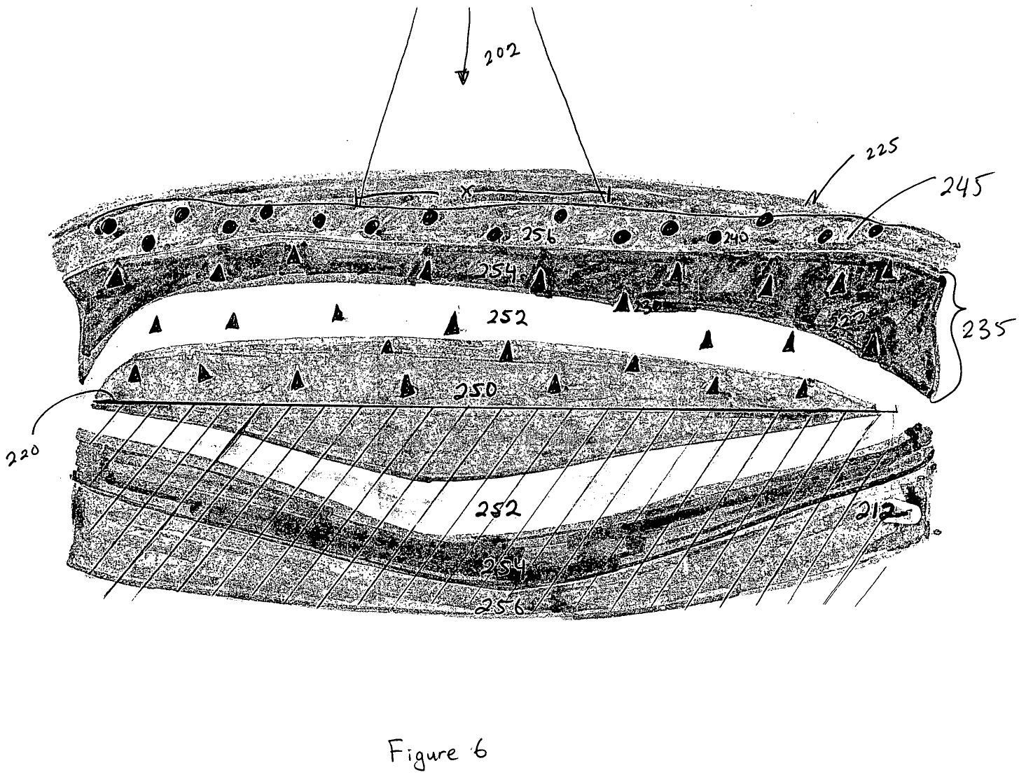

FIG. 6 shows a cross-sectional view of an example of a coating 225 applied to a surface 220 of a strut of a stent 212 that is being exposed to energy 202 from an energy source such as a laser beam generated by a laser. Before being exposed to the energy 202 the coating 225 contained a polymer 222 and only the active form of a therapeutic agent 240. After exposure, the coating 225 contains the polymer 222 and the therapeutic agent in active form 240 and inactive form 230. In order to cause the active form of the therapeutic agent to become inactive, the temperature of the therapeutic agent is raised to its denaturing temperature (Tn).

In this example, it is desirable that the temperature in the parts of the coating that are adjacent to or near the strut to be raise to at least the denaturing temperature of the therapeutic agent so that the therapeutic agent in those parts of the coating becomes inactive while the temperature in other parts of the coating remain below the denaturing temperature so that the therapeutic agent remains in its active form. Therefore, it is desirable to use an energy that is more readily absorbed by the strut 212 than the coating 225. When such an energy is used, the energy 202 is absorbed by the strut 212 and the temperature in the strut will rise. The heat will then travel to the coating by conduction and the temperature of different parts of the coating will rise at different rates and temperature variations in the coated strut will exist, e.g. a temperature gradient in the coating will exist.

In FIG. 6, the different shaded areas defined by the curved lines 250, 252, 254 and 256 indicate thermal contours or temperature gradient within the strut 212 and coating 225 that result from exposure to the energy 202. In the areas designated as 250, 252 and 254, have temperatures at or above the denaturing temperature Tn. In particular, the area designated as 254 is at or at about the denaturing temperature Tn. The area designated as 252 has temperatures above the denaturing temperature and the area designated as 250 has even higher temperatures. On the other hand, the in the area designated as 256 has temperatures below the denaturing temperature. In the region of the coating 235 where the temperatures are at or above the denaturing temperature, the therapeutic agent has become inactivated 230. In the region of the coating 245 where the temperatures are below the denaturing temperature, the therapeutic agent remains in its active form 240.

The surface area, x, of the coating which directly contacts the energy can affect the temperature gradient in the coating. When the energy contacts a relatively small surface area of the coating, the temperature gradient in the coating is steeper due to more localized exposure. When the energy contacts a relatively larger surface area of the coating, the temperature gradient in the coating is less steep since the energy is more widely distributed.

Other factors can also affect the temperature gradient in the coating. These factors include without limitation the intensity of the energy; the ability of the medical device, in this case the strut, to absorb the energy; the thermal conductance of the coating; and the duration in which the coating is exposed to the energy. The temperature gradient in the coating can be modified by adjusting these parameters so that the desired amount of the active form of the therapeutic agent that is raised to the denaturing temperature can be achieved. Also, the steepness of the temperature gradient in the coating can be increased by passing cool air over coating or spraying onto the coating a liquid that readily evaporates but does not dissolve the coating.

Although in the example of FIG. 6 the energy source is shown as being located outside the stent or medical device, in some embodiments, the source can be placed within the device. For example, if the medical device is a stent, the energy-emitting source can be placed within the stent lumen while the energy is being emitted. In another embodiment, the energy source is located outside the stent lumen. In a specific embodiment, the coating is exposed to the activation energy while the medical device is implanted in a patient. In a specific embodiment, the coating is exposed to the activation energy more than once.

In a specific embodiment, the active form of a therapeutic agent may be converted to an inactive form of the therapeutic agent using photochemical bond incision technique. In a specific embodiment, the therapeutic agent comprises a functional group which contributes to the activity of the therapeutic agent. A laser may be used to photochemically cleave the functional group of the therapeutic agent rendering the therapeutic agent inactive.

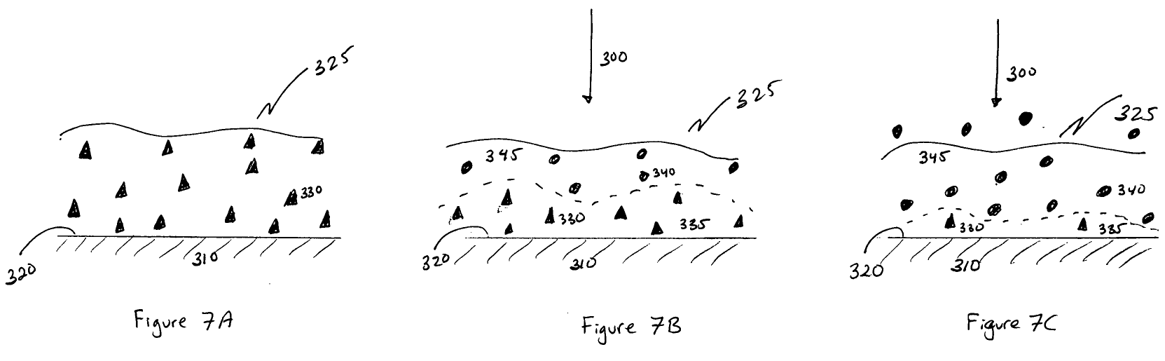

In another embodiment, a coating composition comprising an inactive form of a therapeutic agent 330 is disposed on the surface 320 of a medical device 310 to form a coating 325 as shown in FIG. 7A. The coating 325 is exposed to an activation energy 300 generated by an energy source and the inactive therapeutic agent in a first region 345 becomes an active form of the therapeutic agent 340, i.e. obtains a desired biological or pharmaceutical activity as shown in FIG. 7B. The therapeutic agent in a second region of the coating 335 remains in inactive form 330. When a part or all of the active form of the therapeutic agent 340 has been released from the coating 325, an activation energy 300, which can be the same or different from the previous activation energy, can be applied to the coating 325 to activate the inactive form of the therapeutic agent 330 remaining in the coating 325 as shown in FIG. 7C. In one embodiment, the inactive form of the therapeutic agent remaining in the coating can be activated when the medical device is implanted in a patient. The coating composition can be prepared and applied to surface of the medical device as discussed above.

In a specific embodiment, a prodrug or an inactive form of a therapeutic agent is used as a coating on the surface of a device. In a specific embodiment, the prodrug is a derivative of a biologically active material that can hydrolyze, oxidize, or otherwise react under biological conditions (in vitro or in vivo). These biological conditions include, but are not limited to, exposure to pH, water, temperature or various enzymes. In other embodiments, a laser is used to convert the prodrug or the inactive form of the therapeutic agent to the active form of the therapeutic agent. In a specific embodiment, photochemical bond incision technique is used. In a specific embodiment, the therapeutic agent is attached to a chemical group that renders it inactive. The laser photochemically cleaves the bond that attaches the therapeutic agent to the chemical group. By cleaving the bond between the chemical group and the therapeutic agent, the drug is activated (e.g., Photo Dynamic Therapy as used for skin cancer). To target the region that is close to the surface of the coating, the laser is focused with a very small depth of field for cleaving the bond. In another specific embodiment, the inactive form of a therapeutic agent is activated by heat. The therapeutic agent is encapsulated in a shell that can be melted or degraded at a temperature that will not denature the therapeutic agent within, thereby releasing the therapeutic agent from the coating. In preferred embodiments, the activation energy is generated by an energy source comprising electromagnetic radiation sources including RF and microwave, acoustic wave, ultrasonic wave, ultraviolet laser and infrared laser. In preferred embodiments, for photochemical cleaving of bonds, the wavelengths from 100 nm to 800 nm are preferable. In preferred embodiments, the activation energy is generated by heat using laser on the top surface of the coating having wavelengths from 2 to 20 microns.

Suitable energy sources for activating the inactive form of the therapeutic agent can include without limitation lasers with wavelengths from ultraviolet to far infrared (100 nm to 20,000 nm), RF induction sources, RF electrical sources, microwave sources, ultrasonic sources and heat sources. These energy sources can be applied to the coating for the durations discussed above in connection with the energy sources for causing the active form of a therapeutic agent to become inactive.

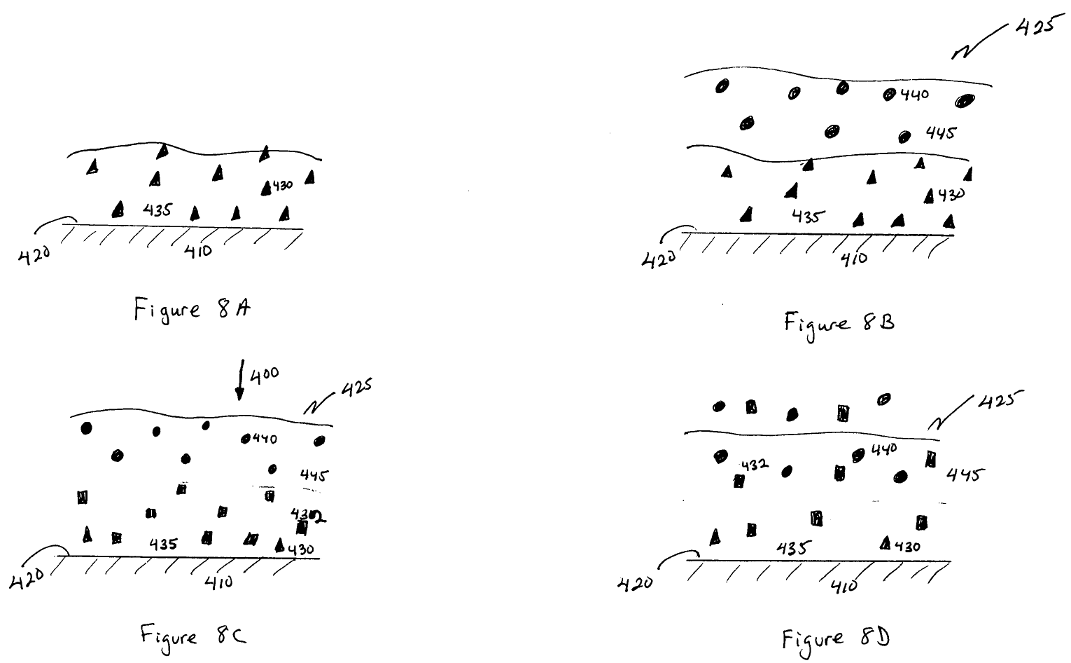

FIGS. 8A-8D depict another embodiment. In this embodiment, a first quantity 435 of a first coating composition comprising an inactive form of a first therapeutic agent 430 is disposed on the surface 420 of a medical device 410 as shown in FIG. 8A. In FIG. 8B a second quantity 445 of a second coating composition comprising an active form of a second therapeutic agent 440, which can be the same or different from the first therapeutic agent, is disposed on the first quantity 435 to form a coating 425. In FIG. 8C, the coating 425 is exposed to an activation energy 400 from an energy source to activate the inactive form of the first therapeutic agent 430, which results in an active form of the first therapeutic agent 432. Preferably, the inactive form of the first therapeutic agent 430 is activated after at least a portion of the active form of the second therapeutic agent 440 has been released from the coating 425. Also, preferably, the exposure to the energy occurs while the medical device is implanted in patient. FIG. 8D shows the active form of the first therapeutic agent 432 being released from the coating 425 after exposure to the energy 400. The coating compositions can be prepared and applied to surface of the medical device as discussed above. Also, the energy sources for activating a therapeutic agent that were discussed above are suitable for this embodiment.

### 5.3. Therapeutic Agents

In certain embodiments, the therapeutic agent is useful for inhibiting cell proliferation, contraction, migration, hyperactivity, or addressing other conditions such as cancer.

The term “therapeutic agent” as used in the present invention encompasses drugs or pharmaceuticals, genetic materials, and biological materials and can be used interchangeably with “biologically active material”. Non-limiting examples of suitable therapeutic agent include heparin, heparin derivatives, urokinase, dextrophenylalanine proline arginine chloromethylketone (PPack), enoxaparin, angiopeptin, hirudin, acetylsalicylic acid, tacrolimus, everolimus, rapamycin (sirolimus), pimecrolimus, amlodipine, doxazocin, glucocorticoids, betamethasone, dexamethasone, prednisolone, corticosterone, budesonide, sulfasalazine, rosiglitazone, mycophenolic acid, mesalamine, paclitaxel, 5-fluorouracil, cisplatin, vinblastine, vincristine, epothilones, methotrexate, azathioprine, adriamycin, mutamycin, endostatin, angiostatin, thymidine kinase inhibitors, cladribine, lidocaine, bupivacaine, ropivacaine, D-Phe-Pro-Arg chloromethyl ketone, platelet receptor antagonists, anti-thrombin antibodies, anti-platelet receptor antibodies, aspirin, dipyridamole, protamine, hirudin, prostaglandin inhibitors, platelet inhibitors, trapidil, liprostin, tick antiplatelet peptides, 5-azacytidine, vascular endothelial growth factors, growth factor receptors, transcriptional activators, translational promoters, antiproliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin, cholesterol lowering agents, vasodilating agents, agents which interfere with endogenous vasoactive mechanisms, antioxidants, probucol, antibiotic agents, penicillin, cefoxitin, oxycillin, tobramycin, angiogenic substances, fibroblast growth factors, estrogen, estradiol (E2), estriol (E3), 17-beta estradiol, digoxin, beta blockers, captopril, enalapril, statins, steroids, vitamins, paclitaxel (as well as its derivatives, analogs or paclitaxel bound to proteins, e.g. Abraxane™) 2′-succinoyl-taxol, 2′-succinoyl-taxol triethanolamine, 2′-glutaryl-taxol, 2′-glutaryl-taxol triethanolamine salt, 2′-O-ester with N-(dimethylaminoethyl) glutamine, 2′-O-ester with N-(dimethylaminoethyl) glutamide hydrochloride salt, nitroglycerin, nitrous oxides, nitric oxides, antibiotics, aspirins, digitalis, estrogen, estradiol and glycosides. In one embodiment, the therapeutic agent is a smooth muscle cell inhibitor or antibiotic. In one preferred embodiment, the therapeutic agent is an antibiotic such as erythromycin, amphotericin, rapamycin, adriamycin, etc.

The term “genetic materials” means DNA or RNA, including, without limitation, of DNA/RNA encoding a useful protein stated below, intended to be inserted into a human body including viral vectors and non-viral vectors.

The term “biological materials” include cells, yeasts, bacteria, proteins, peptides, cytokines and hormones. Examples for peptides and proteins include vascular endothelial growth factor (VEGF), transforming growth factor (TGF), fibroblast growth factor (FGF), epidermal growth factor (EGF), cartilage growth factor (CGF), nerve growth factor (NGF), keratinocyte growth factor (KGF), skeletal growth factor (SGF), osteoblast-derived growth factor (BDGF), hepatocyte growth factor (HGF), insulin-like growth factor (IGF), cytokine growth factors (CGF), platelet-derived growth factor (PDGF), hypoxia inducible factor-1 (HIF-1), stem cell derived factor (SDF), stem cell factor (SCF), endothelial cell growth supplement (ECGS), granulocyte macrophage colony stimulating factor (GM-CSF), growth differentiation factor (GDF), integrin modulating factor (IMF), calmodulin (CaM), thymidine kinase (TK), tumor necrosis factor (TNF), growth hormone (GH), bone morphogenic protein (BMP) (e.g., BMP-2, BMP-3, BMP-4, BMP-5, BMP-6 (Vgr-1), BMP-7 (PO-1), BMP-8, BMP-9, BMP-10, BMP-11, BMP-12, BMP-14, BMP-15, BMP-16, etc.), matrix metalloproteinase (MMP), tissue inhibitor of matrix metalloproteinase (TIMP), cytokines, interleukin (e.g., IL-1, IL-2, IL-3, IL-4, IL-5, IL-6, IL-7, IL-8, IL-9, IL-10, IL-11, IL-12, IL-15, etc.), lymphokines, interferon, integrin, collagen (all types), elastin, fibrillins, fibronectin, vitronectin, laminin, glycosaminoglycans, proteoglycans, transferrin, cytotactin, cell binding domains (e.g., RGD), and tenascin. Currently preferred BMP's are BMP-2, BMP-3, BMP-4, BMP-5, BMP-6, BMP-7. These dimeric proteins can be provided as homodimers, heterodimers, or combinations thereof, alone or together with other molecules. Cells can be of human origin (autologous or allogeneic) or from an animal source (xenogeneic), genetically engineered, if desired, to deliver proteins of interest at the transplant site. The delivery media can be formulated as needed to maintain cell function and viability. Cells include progenitor cells (e.g., endothelial progenitor cells), stem cells (e.g., mesenchymal, hematopoietic, neuronal), stromal cells, parenchymal cells, undifferentiated cells, fibroblasts, macrophage, and satellite cells.

Other non-genetic therapeutic agents include:
anti-thrombogenic agents such as heparin, heparin derivatives, urokinase, and PPack (dextrophenylalanine proline arginine chloromethylketone);
anti-proliferative agents such as enoxaparin, angiopeptin, or monoclonal antibodies capable of blocking smooth muscle cell proliferation, hirudin, acetylsalicylic acid, tacrolimus, everolimus, amlodipine and doxazocin;
anti-inflammatory agents such as glucocorticoids, betamethasone, dexamethasone, prednisolone, corticosterone, budesonide, estrogen, sulfasalazine, rosiglitazone, mycophenolic acid and mesalamine;
anti-neoplastic/anti-proliferative/anti-miotic agents such as paclitaxel, 5-fluorouracil, cisplatin, vinblastine, vincristine, epothilones, methotrexate, azathioprine, adriamycin and mutamycin; endostatin, angiostatin and thymidine kinase inhibitors, cladribine, taxol and its analogs or derivatives;
anesthetic agents such as lidocaine, bupivacaine, and ropivacaine;
anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, aspirin (aspirin is also classified as an analgesic, antipyretic and anti-inflammatory drug), dipyridamole, protamine, hirudin, prostaglandin inhibitors, platelet inhibitors, antiplatelet agents such as trapidil or liprostin and tick antiplatelet peptides;
DNA demethylating drugs such as 5-azacytidine, which is also categorized as a RNA or DNA metabolite that inhibit cell growth and induce apoptosis in certain cancer cells;
vascular cell growth promoters such as growth factors, vascular endothelial growth factors (VEGF, all types including VEGF-2), growth factor receptors, transcriptional activators, and translational promoters;
vascular cell growth inhibitors such as anti-proliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin;
cholesterol-lowering agents, vasodilating agents, and agents which interfere with endogenous vasoactive mechanisms;
anti-oxidants, such as probucol;
antibiotic agents, such as penicillin, cefoxitin, oxycillin, tobramycin, rapamycin (sirolimus);
angiogenic substances, such as acidic and basic fibroblast growth factors, estrogen including estradiol (E2), estriol (E3) and 17-beta estradiol;
drugs for heart failure, such as digoxin, beta-blockers, angiotensin-converting enzyme (ACE) inhibitors including captopril and enalapril, statins and related compounds;
macrolides such as sirolimus or everolimus; and
anti-restenotic agents.

Preferred biological materials include anti-proliferative drugs such as steroids, vitamins, and restenosis-inhibiting agents. Preferred restenosis-inhibiting agents include microtubule stabilizing agents such as paclitaxel (i.e., paclitaxel, paclitaxel analogs, or paclitaxel derivatives, and mixtures thereof). For example, derivatives suitable for use in the present invention include 2′-succinyl-taxol, 2′-succinyl-taxol triethanolamine, 2′-glutaryl-taxol, 2′-glutaryl-taxol triethanolamine salt, 2′-O-ester with N-(dimethylaminoethyl) glutamine, and 2′-O-ester with N-(dimethylaminoethyl) glutamide hydrochloride salt.

Other suitable therapeutic agents include tacrolimus; halofuginone; inhibitors of HSP90 heat shock proteins such as geldanamycin; microtubule stabilizing agents such as epothilone D; phosphodiesterase inhibitors such as cilostazole; Barket inhibitors; phospholamban inhibitors; and Serca 2 gene/proteins.

Other preferred therapeutic agents include nitroglycerin, nitrous oxides, nitric oxides, aspirins, digitalis, estrogen derivatives such as estradiol and glycosides.

In one embodiment, the therapeutic agent is capable of altering the cellular metabolism or inhibiting a cell activity, such as protein synthesis, DNA synthesis, spindle fiber formation, cellular proliferation, cell migration, microtubule formation, microfilament formation, extracellular matrix synthesis, extracellular matrix secretion, or increase in cell volume. In another embodiment, the therapeutic agent is capable of inhibiting cell proliferation and/or migration. In certain embodiments, the therapeutic agent is capable of inhibiting the proliferation contraction, migration or hyperactivity of cells, such as smooth muscle cells.

In one embodiment, one or more therapeutic agents may be encapsulated. The therapeutic agent can be encapsulated by methods well known to one skilled in the art (see, e.g., Radtchenko et al., A novel method for encapsulation of poorly water-soluble drugs: precipitation in polyelectrolyte multilayer shells. Int J. Pharm. 2002; 242: 219-23; Antipov et al. Polyelectrolyte multilayer capsule permeability control. Colloids and Surfaces A: Physiocochem Eng Aspects 2002; 198-200: 535-541; Qiu et al. Studies on the drug release properties of polysaccharide multilayers encapsulated ibuprofen microparticles. Langmuir 2001; 17: 5375-5380; Moya et al. Polyelectrolyte multilayer capsules templated on biological cells: core oxidation influences layer chemistry. Colloids and Surfaces A: Physiocochem Eng Aspects 2001; 183-185: 27-40; Radtchenko et al. Assembly of Alternated Multivalent Ion/Polyelectrolyte Layers on Colloidal Particles. Stability of the Multilayers and Encapsulation of Macromolecules into Polyelectrolyte Capsules. J Colloid Interface Sci. 2000; 230: 272-280; Voigt et al. Membrane filtration for microencapsulation and microcapsules fabrication by layer-by-layer polyelectrolyte adsorption. Ind Eng Chem Res. 1999; 38: 4037-4043; Donath et al. Novel hollow polymer shells: fabrication, characterization and potential applications. Angewandte Chemie 1998; 37: 2201-2205; International Publication No. WO 95/08320; and U.S. Pat. No. 6,322,817 issued to Maitra et al. and U.S. Pat. No. 6,007,845 issued to Domb et al., each of which is incorporated by reference herein in its entirety).

In specific embodiments, the therapeutic agent is in the form of a prodrug. In specific embodiments, the therapeutic agent is a cell-targeting molecule comprising a cell-targeting portion and a biologically active portion. In particular, the cell-targeting portion provides selective targeting of a particular cell type, e.g., disease-associated cells, via monoclonal antibodies or other cell-specific molecules that bind such diseased-cell specific proteins. Examples of cell-targeting portion includes, but are not limited to, cell surface molecules, members of a binding pair (such as a ligand or a receptor), growth factors or antigen-binding domains of antibodies, including the Fv portion of an antibody or single-chain antibodies, that are fused or conjugated to various biological materials as discussed above.

### 5.4 Polymers

As used herein, the term “polymer” is used interchangeable with the terms “polymer material” and “polymeric matrix”. Polymers suitable for use in the preparation of the coatings of the present invention should be a material that is biocompatible and avoids irritation to body tissue. Preferably, the polymer used in the coating compositions of the present invention are selected from the following: ethylene vinyl acetate, polybutyl methacrylate, polyurethanes, silicones (e.g., polysiloxanes and substituted polysiloxanes), and polyesters. Also preferable as a polymeric material is copolymers of styrene and isobutylene. Other polymers which can be used include ones that can be dissolved and cured or polymerized on the medical device or polymers having relatively low melting points that can be blended with biologically active materials. Additional suitable polymers include, thermoplastic elastomers in general, polyolefins, polyisobutylene, ethylene-alphaolefin copolymers, acrylic polymers and copolymers, vinyl halide polymers and copolymers such as poly(lactide-co-glycolide) (PLGA), polyvinyl alcohol (PVA), poly(L-lactide) (PLLA), polyanhydrides, polyphosphazenes, polycaprolactone (PCL), polyvinyl chloride, polyvinyl ethers such as polyvinyl methyl ether, polyvinylidene halides such as polyvinyl idene fluoride and polyvinylidene chloride, polyacrylonitrile, polyvinyl ketones, polyvinyl aromatics such as polystyrene, polyvinyl esters such as polyvinyl acetate, copolymers of vinyl monomers, copolymers of vinyl monomers and olefins such as ethylene-methyl methacrylate copolymers, acrylonitrile-styrene copolymers, ABS (acrylonitrile-butadiene-styrene) resins, ethylene-vinyl acetate copolymers, polyamides such as Nylon 66 and polycaprolactone, alkyd resins, polycarbonates, polyoxymethylenes, polyimides, polyethers, epoxy resins, rayon-triacetate, cellulose, cellulose acetate, cellulose butyrate, cellulose acetate butyrate, cellophane, cellulose nitrate, cellulose propionate, cellulose ethers, carboxymethyl cellulose, collagens, chitins, polylactic acid (PLA), polyglycolic acid (PGA), polyethylene oxide (PEO), polylactic acid-polyethylene oxide copolymers, EPDM (etylene-propylene-diene) rubbers, fluorosilicones, polyethylene glycol (PEG), polyalkylene glycol (PAG), polysaccharides, phospholipids, and combinations of the foregoing.

In certain embodiments, the polymer is hydrophilic (e.g., PVA, PLLA, PLGA, PEG, and PAG). In certain other embodiments, the polymer is hydrophobic (e.g., PLA, PGA, polyanhydrides, polyphosphazenes, PCL, copolymers of styrene and isobutylene, and polyorthoesters).

More preferably for medical devices which undergo mechanical challenges, e.g., expansion and contraction, the polymer should be selected from elastomeric polymers such as silicones (e.g., polysiloxanes and substituted polysiloxanes), polyurethanes, thermoplastic elastomers, ethylene vinyl acetate copolymers, polyolefin elastomers, and EPDM rubbers. Because of the elastic nature of these polymer, the coating composition is capable of undergoing deformation under the yield point when the device is subjected to forces, stress or mechanical challenge.

The polymer may be biodegradable or biostable. In some embodiments, the polymer is biodegradable. Biodegradable polymeric materials can degrade as a result of hydrolysis of the polymer chains into biologically acceptable, and progressively smaller compounds. In one embodiment, a polymeric material comprises polylactides, polyglycolides, or their co-polymers. Polylactides, polyglycolides, and their co-polymers break down to lactic acid and glycolic acid, which enters the Kreb's cycle and are further broken down into carbon dioxide and water.

Biodegradable solids may have differing modes of degradation. On one hand, degradation by bulk erosion/hydrolysis occurs when water penetrates the entire structure and degrades the entire structure simultaneously, i.e., the polymer degrades in a fairly uniform manner throughout the structure. On the other hand, degradation by surface erosion occurs when degradation begins from the exterior with little/no water penetration into the bulk of the structure (see, e.g., Gopferich A. Mechanisms of polymer degradation and erosion. Biomaterials 1996; 17(103):243-259, which is incorporated by reference herein in its entirety). For some novel degradable polymers, most notably the polyanhydrides and polyorthoesters, the degradation occurs only at the surface of the polymer, resulting in a release rate that is proportional to the surface area of the drug delivery system. Hydrophilic polymeric materials such as PLGA will erode in a bulk fashion. Various commercially available PLGA may be used in the preparation of the coating compositions. For example, poly(d,1-lactic-co-glycolic acid) are commercially available. A preferred commercially available product is a 50:50 poly (D,L) lactic co-glycolic acid having a mole percent composition of 50% lactide and 50% glycolide. Other suitable commercially available products are 65:35 DL, 75:25 DL, 85:15 DL and poly(d,1-lactic acid) (d,1-PLA). For example, poly(lactide-co-glycolides) are also commercially available from Boehringer Ingelheim (Germany) under its Resomer©, e.g., PLGA 50:50 (Resomer RG 502), PLGA 75:25 (Resomer RG 728) and d,1-PLA (resomer RG 206), and from Birmingham Polymers (Birmingham, Ala.). These copolymers are available in a wide range of molecular weights and ratios of lactic to glycolic acid.

In one embodiment, the coating comprises copolymers with desirable hydrophilic/hydrophobic interactions (see, e.g., U.S. Pat. No. 6,007,845, which describes nanoparticles and microparticles of non-linear hydrophilic-hydrophobic multiblock copolymers, which is incorporated by reference herein in its entirety). In a specific embodiment, the coating comprises ABA triblock copolymers consisting of biodegradable A blocks from PLG and hydrophilic B blocks from PEO.

### 5.5 Non-Polymeric Materials

The coating compositions suitable for the present invention can include non-polymeric materials. The non-polymeric material suitable for use in the preparation of the coatings of the present invention should be a material that is biocompatible and avoids irritation to body tissue. Preferably, the non-polymeric materials used in the coating compositions of the present invention are selected from the following: sterols such as cholesterol, stigmasterol, beta-sitosterol, and estradiol; cholesteryl esters such as cholesteryl stearate; C₁₂-C₂₄ fatty acids such as lauric acid, myristic acid, palmitic acid, stearic acid, arachidic acid, behenic acid, and lignoceric acid; C₁₈-C₃₆ mono-, di- and triacylglycerides such as glyceryl monooleate, glyceryl monolinoleate, glyceryl monolaurate, glyceryl monodocosanoate, glyceryl monomyristate, glyceryl monodicenoate, glyceryl dipalmitate, glyceryl didocosanoate, glyceryl dimyristate, glyceryl didecenoate, glyceryl tridocosanoate, glyceryl trimyristate, glyceryl tridecanoate, glycerol tristearate and mixtures thereof; sucrose fatty acid esters such as sucrose distearate and sucrose palmitate; sorbitan fatty acid esters such as sorbitan monostearate, sorbitan monopalmitate and sorbitan tristearate; C₁₆-C₁₈ fatty alcohols such as cetyl alcohol, myristyl alcohol, stearyl alcohol, and cetostearyl alcohol; esters of fatty alcohols and fatty acids such as cetyl palmitate and cetearyl palmitate; anhydrides of fatty acids such as stearic anhydride; phospholipids including phosphatidylcholine (lecithin), phosphatidylserine, phosphatidylethanolamine, phosphatidylinositol, and lysoderivatives thereof; sphingosine and derivatives thereof; sphingomyelins such as stearyl, palmitoyl, and tricosanyl sphingomyelins; ceramides such as stearyl and palmitoyl ceramides; glycosphingolipids; lanolin and lanolin alcohols; and combinations and mixtures thereof. Preferred non-polymeric materials include cholesterol, glyceryl monostearate, glycerol tristearate, stearic acid, stearic anhydride, glyceryl monooleate, glyceryl monolinoleate, and acetylated monoglycerides.

In certain embodiments, the non-polymeric material is hydrophilic. In a specific embodiment, the hydrophilic non-polymeric material comprises myristyl alcohol. In another specific embodiment, the hydrophilic non-polymeric material comprises carbon structures such as carbon tubes or balls, which can be made hydrophilic by attaching carboxylic acid groups by means of an acid treatment.

In certain other embodiments, the non-polymeric material is hydrophobic. In a specific embodiment, the hydrophobic non-polymeric material comprises cholesterol. In another specific embodiment, the hydrophobic non-polymeric material comprises liposomes.

In preferred embodiments, the non-polymeric materials can undergo forces, stress or mechanical challenges, e.g., expansion and contraction.

In preferred embodiments, the non-polymeric materials are biodegradable.

In certain preferred embodiments, the therapeutic agents described in Section 5.1.1.1 supra. are mixed with one or more polymers. Such mixture can be used to form a medical device or portions thereof. In specific embodiments, the therapeutic agent and/or coating compositions comprising the therapeutic agent constitute at least 5%, at least 10%, at least 20%, at least 30%, at least 40%, at least 50%, at least 60%, at least 70%, at least 80%, at least 90%, at least 95%, at least 97%, at least 99% or more by weight of the polymeric materials used to form the medical device.

### 5.6 Types of Medical Devices

Preferred examples of the medical devices suitable for the present invention include, but are not limited to, stents, surgical staples, catheters (e.g., central venous catheters and arterial catheters), guidewires, cannulas, cardiac pacemaker leads or lead tips, cardiac defibrillator leads or lead tips, implantable vascular access ports, blood storage bags, blood tubing, vascular or other grafts, intra-aortic balloon pumps, heart valves, cardiovascular sutures, total artificial hearts and ventricular assist pumps, and extra-corporeal devices such as blood oxygenators, blood filters, hemodialysis units, hemoperfusion units and plasmapheresis units. In a preferred embodiment, the medical device is a stent.

Medical devices of the present invention include those that have a tubular or cylindrical-like portion. The tubular portion of the medical device need not be completely cylindrical. For instance, the cross-section of the tubular portion can be any shape, such as rectangle, a triangle, etc., not just a circle. Such devices include, without limitation, stents and grafts. A bifurcated stent is also included among the medical devices which can be fabricated by the method of the present invention.

In addition, the tubular portion of the medical device may be a sidewall that is comprised of a plurality of struts defining a plurality of openings or an open lattice structure. The struts may be arranged in any suitable configuration. Also, the struts do not all have to have the same shape or geometric configuration. Each individual strut has a surface adapted for exposure to the body tissue of the patient. In preferred embodiments, the medical device is a stent that comprises a tubular body having open ends and an open lattice sidewall structure and the coating conforms to the sidewall structure.

The medical device may be formed after application of the coating or it may be pre-fabricated before application of the coating. The pre-fabricated medical device is in its final shape. For example, if the finished medical device is a stent having an opening in its sidewall, then the opening is formed in the device before application of the coating.

Medical devices which are particularly suitable for the present invention include any kind of stent for medical purposes which is known to the skilled artisan. Suitable stents include, for example, vascular stents such as self-expanding stents and balloon expandable stents. Examples of self-expanding stents useful in the present invention are illustrated in U.S. Pat. No. 4,655,771 and 4,954,11 issued to Wallsten and U.S. Pat. No. 5,061,275 issued to Wallsten et al. Examples of appropriate balloon-expandable stents are shown in U.S. Pat. No. 5,449,373 issued to Pinchasik et al.

Medical devices that are useful in the present invention can be made of any biocompatible material suitable for medical devices in general which include without limitation natural polymers, synthetic polymers, ceramics, and metallics. In certain embodiments, ceramic material is preferred. Suitable ceramic materials include, but are not limited to, oxides, carbides, or nitrides of the transition elements such as titaniumoxides, hafnium oxides, iridiumoxides, chromium oxides, aluminum oxides, and zirconiumoxides. Silicon based materials, such as silica, may also be used. In certain other embodiments, metallic material (e.g., niobium, niobium-zirconium, and tantalum) is more preferable. Suitable metallic materials include metals and alloys based on titanium (such as nitinol, nickel titanium alloys, thermo-memory alloy materials), stainless steel, tantalum, nickel-chrome, or certain cobalt alloys including cobalt-chromium-nickel alloys such as Elgiloy® and Phynox®. Metallic materials also include clad composite filaments, such as those disclosed in WO 94/16646.

Metallic materials may be made into elongated members or wire-like elements and then woven to form a network of metal mesh. Polymer filaments may also be used together with the metallic elongated members or wire-like elements to form a network mesh. If the network is made of metal, the intersection may be welded, twisted, bent, glued, tied (with suture), heat sealed to one another; or connected in any manner known in the art.

The polymer(s) useful for forming the medical device should be ones that are biocompatible and avoid irritation to body tissue. They can be either biostable or bioabsorbable. Suitable polymeric materials include without limitation polyurethane and its copolymers, silicone and its copolymers, ethylene vinyl-acetate, polyethylene terephthalate, thermoplastic elastomers, polyvinyl chloride, polyolefins, cellulosics, polyamides, polyesters, polysulfones, polytetrafluorethylenes, polycarbonates, acrylonitrile butadiene styrene copolymers, acrylics, polylactic acid, polyglycolic acid, polycaprolactone, polylactic acid-polyethylene oxide copolymers, cellulose, collagens, and chitins.

Other polymers that are useful as materials for medical devices include without limitation dacron polyester, poly(ethylene terephthalate), polycarbonate, polymethylmethacrylate, polypropylene, polyalkylene oxalates, polyvinylchloride, polyurethanes, polysiloxanes, nylons, poly(dimethyl siloxane), polycyanoacrylates, polyphosphazenes, poly(amino acids), ethylene glycol I dimethacrylate, poly(methyl methacrylate), poly(2-hydroxyethyl methacrylate), polytetrafluoroethylene poly(HEMA), polyhydroxyalkanoates, polytetrafluroethylene, polycarbonate, poly(glycolide-lactide) co-polymer, polylactic acid, poly(ÿ-caprolactone), poly(ÿ-hydroxybutyrate), polydioxanone, poly(ÿ-ethyl glutamate), polyiminocarbonates, poly(ortho ester), polyanhydrides, alginate, dextran, chitin, cotton, polyglycolic acid, polyurethane, or derivatized versions thereof, i.e., polymers which have been modified to include, for example, attachment sites or cross-linking groups, e.g., Arg-Gly-Asp (RGD), in which the polymers retain their structural integrity while allowing for attachment of molecules, such as proteins, nucleic acids, and the like.

The polymers may be dried to increase its mechanical strength. The polymers may then be used as the base material to form a whole or part of the medical device.

Furthermore, although the invention can be practiced by using a single type of polymer to form the medical device, various combinations of polymers can be employed. The appropriate mixture of polymers can be coordinated to produce desired effects when incorporated into a medical device.

In a specific embodiment, the medical device comprises a surface comprising a ceramic layer. Preferably, the ceramic layer extends the time period for releasing the therapeutic agent from the medical device.

The therapeutic agent may also be used to form the medical device. In one embodiment, the therapeutic agent may be incorporated into the base material needed to make the device.

### 5.7. Therapeutic Uses

The coated medical devices of the present invention can be used to treat or prevent diseases or conditions in a mammal. Such coated medical devices can be used in combination with other forms of treatment or prevention.

In certain embodiments, coated medical devices of the present invention may be used to inhibit the proliferation, contraction, migration and/or hyperactivity of cells of the brain, neck, eye, mouth, throat, esophagus, chest, bone, ligament, cartilage, tendons, lung, colon, rectum, stomach, prostate, breast, ovaries, fallopian tubes, uterus, cervix, testicles or other reproductive organs, hair follicles, skin, diaphragm, thyroid, blood, muscles, bone, bone marrow, heart, lymph nodes, blood vessels, arteries, capillaries, large intestine, small intestine, kidney, liver, pancreas, brain, spinal cord, and the central nervous system. In a preferred embodiment, the coated medical devices are useful for inhibiting the proliferation, contraction, migration and/or hyperactivity of muscle cells, e.g., smooth muscle cells.

In certain other embodiments, the coated medical devices of the present invention may be used to inhibit the proliferation, contraction, migration and/or hyperactivity of cells in body tissues, e.g., epithelial tissue, connective tissue, muscle tissue, and nerve tissue. Epithelial tissue covers or lines all body surfaces inside or outside the body. Examples of epithelial tissue include, but are not limited to, the skin, epithelium, dermis, and the mucosa and serosa that line the body cavity and internal organs, such as the heart, lung, liver, kidney, intestines, bladder, uterine, etc. Connective tissue is the most abundant and widely distributed of all tissues. Examples of connective tissue include, but are not limited to, vascular tissue (e.g., arteries, veins, capillaries), blood (e.g., red blood cells, platelets, white blood cells), lymph, fat, fibers, cartilage, ligaments, tendon, bone, teeth, omentum, peritoneum, mesentery, meniscus, conjunctiva, dura mater, umbilical cord, etc. Muscle tissue accounts for nearly one-third of the total body weight and consists of three distinct subtypes: striated (skeletal) muscle, smooth (visceral) muscle, and cardiac muscle. Examples of muscle tissue include, but are not limited to, myocardium (heart muscle), skeletal, intestinal wall, etc. The fourth primary type of tissue is nerve tissue. Nerve tissue is found in the brain, spinal cord, and accompanying nerve. Nerve tissue is composed of specialized cells called neurons (nerve cells) and neuroglial or glial cells.

The coated medical devices of the present invention may also be used to treat diseases that may benefit from decreased cell proliferation, contraction, migration and/or hyperactivity, including, but not limited to stenosis, restenosis and cancer.

The present invention is not to be limited in scope by the specific embodiments described which are intended as single illustrations of individual aspects of the invention, and functionally equivalent methods and components are within the scope of the invention. Indeed, various modifications of the invention, in addition to those shown and described herein, will become apparent to those skilled in the art from the foregoing description and accompanying drawings using no more than routine experimentation. Such modifications and equivalents are intended to fall within the scope of the appended claims.

All publications, patents and patent applications mentioned in this specification are herein incorporated by reference in their entirety into the present specification to the same extent as if each individual publication, patent or patent application was specifically and individually indicated to be incorporated herein by reference.

What is claimed is:

1. A medical device comprising:
a surface; and
a coating disposed on at least a portion of the surface, wherein the coating comprises a first region comprising a first amount of an inactive form of a first therapeutic agent and a second region situated over at least a portion of the first region, wherein the second region comprises a second amount of an active form of a second therapeutic agent.

2. The medical device of claim 1, wherein the second region is situated adjacent to the first region.

3. The medical device of claim 1, wherein the first and second therapeutic agents are the same.

4. The medical device of claim 1, wherein the first and second therapeutic agents are different.

5. The medical device of claim 1, wherein the first region comprises the active form of the second therapeutic agent in an amount of less than 1 weight percent of the first region.

6. The medical device of claim 1, wherein the second region comprises the inactive form of the first therapeutic agent in an amount of less than 1 weight percent of the second region.

7. The medical device of claim 1, wherein the coating is capable of providing sustained release of the active form of the second therapeutic agent over a period of about 1 month to about 1 year.

8. The medical device of claim 1, wherein the coating is capable of providing sustained release of the active form of the second therapeutic agent over a period of about 1 month to about 6 months.

9. The medical device of claim 1 wherein the coating further comprises a third region.

10. The medical device of claim 9, wherein the third region is situated between the first region and the second region, and wherein the third region comprises the active form of the second therapeutic agent and the inactive form of the first therapeutic agent.

11. The medical device of claim 9, wherein the third region comprises the active form of the second therapeutic agent in an amount of less than 1 weight percent of the third region.

12. The medical device of claim 1, wherein the first region is a first layer and the second region is a second layer situated over at least a portion of the first layer.

13. The medical device of claim 12, wherein the coating further comprises a third layer.

14. The medical device of claim 13, wherein the third layer is situated between the first and second layers.

15. The medical device of claim 1, wherein at least one of the first or the second therapeutic agent inhibits smooth muscle cell proliferation, contraction, migration or hyperactivity.

16. The medical device of claim 1, wherein at least one of the first or the second therapeutic agent comprises an antibiotic, an immunosuppressant, or an antiproliferative agent.

17. The medical device of claim 1 wherein at least one of the first or the second therapeutic agent comprises sirolimus, everolimus, tacrolimus, or pimecrolimus.

18. The medical device of claim 1, wherein at least one of the first or the second therapeutic agent comprises paclitaxel.

19. The medical device of claim 1, wherein the coating comprises a polymer.

20. The medical device of claim 19, wherein said polymer comprises a(n) ethylene vinyl acetate, polybutyl methacrylate, styrene-isobutylene-styrene, polyurethane, silicone, polyester, polyolefin, polyisobutylene, ethylene-alphaolefin copolymer, acrylic polymer or acrylic copolymer, vinyl halide polymer, polyvinyl ether, polyvinylidene halide, polyacrylonitrile, polyvinyl ketone, polyvinyl aromatic, polyvinyl ester, copolymer of vinyl monomer, copolymer of vinyl monomer and olefin, polyamide, alkyd resin, polycarbonate, polyoxymethylene, polyimide, polyether, epoxy resin, polyurethane, rayon-triacetate, cellulose, cellulose acetate, cellulose butyrate, cellulose acetate butyrate, cellophane, cellulose nitrate, cellulose propionate, cellulose ether, carboxymethyl cellulose, collagen, chitin, polylactic acid, polyglycolic acid, polylactic acid-polyethylene oxide copolymer, EPDM rubber, fluorosilicone, polyethylene glycol, polysaccharide, or phospholipid.

21. The medical device of claim 1, wherein the first region comprises a polymer.

22. The medical device of claim 1, wherein the second region comprises a polymer.

23. The medical device of claim 1 wherein the first region comprises a first polymer and the second region comprises a second polymer.

24. The medical device of claim 23, wherein the first polymer and the second polymer are different.

25. The medical device of claim 24, wherein the first region further comprises a third polymer and the third polymer is the same as the second polymer.

26. An implantable stent comprising:
a metallic intravascular, balloon-expandable open lattice sidewall stent structure designed for permanent implantation into a blood vessel of a patient; and
a coating conforming to the open lattice sidewall so as to preserve the open lattice sidewall structure of the stent, wherein the coating comprises a first region comprising a first amount of an inactive form of a therapeutic agent and a second region situated over at least a portion of the first region, wherein the second region comprises a second amount of an active form of the therapeutic agent and wherein the therapeutic agent inhibits smooth muscle cell proliferation, contraction, migration or hyperactivity.
